(19) United States
(12) Patent Application Publication
(10) Pub. No.: US 2003/0033638 A1
(43) Pub. Date: Feb. 13, 2003

(54) Production of transgenic plants of the tagetes species

(76) Inventors: Irene Kunze, Gatersleben (DE); Karin Herbers, Quedlinburg (DE); Ute Heim, Gatersleben (DE)

Correspondence Address: Nixon & Vanderhye, 8th Floor, 1100 North Glebe Road, Arlington, VA, 22201-4714, US

(21) Appl. No.: 10/168,018

(22) PCT Filed: Dec. 13, 2000
(86) PCT No.: PCT/EP00/12643

(30) Foreign Application Priority Data
Dec. 21, 1999 (DE) 19962133.0

(51) Int. Cl. A01H 1/00; C12N 15/82

(52) U.S. Cl. 800/294; 435/469

(57) ABSTRACT

A method of producing transgenic plants of the genus Tagetes which is based on an Agrobacterium-tumefaciens-mediated transformation of cell cultures, the subsequent selection of the transformed cells and their regeneration into stably transformed plants is described. Transformed cells and regenerated transgenic plants express the foreign gene.

The invention relates to a method for the generation of stably transformed fertile plants of the genus Tagetes, which comprises the following steps:

(a) growing the starting plant to be transformed, and obtaining the suitable explant, (b) transferring DNA sequences into plant cells, (c) selecting transformed plant cells, and (d) regenerating transgenic fertile plants. Moreover, the method comprises the possibility of transferring DNA sequences into the plant to be transformed or explants thereof, for example by Agrobacterium tumefaciens. Moreover, the method comprises the possibility of using leaves as explants, of carrying out a cocultivation with Agrobacterium for 2 to 8 days and of using during the cocultivation a medium which contains growth regulators. The process furthermore comprises the possibility that, for example, phosphinothricin can be employed for the selection and that two different combinations of growth regulators are used for the regeneration into intact transgenic plants. The method is suitable for transforming the species Tagetes erecta or Tagetes patula. The invention also relates to transgenic fertile Tagetes plants themselves which have been generated by the method described, and to their transgenic seeds.

The plant genus Tagetes, which belongs to the family of the Asteraceae, exhibits quite a few interesting properties, which explains its use both as a crop plant and as an ornamental. Many species of this genus produce bioactive compounds with a nematicidal, fungicidal and insecticidal action, accumulate flavonoids and carotenoids with antioxidant and coloring activities, are salt resistent, and serve decorative purposes owing to their wide range of flower colors and flower shapes.

The nematicidal action of the Tagetes roots is based on the synthesis of thiophene derivatives. Thiophene derivatives are heterocyclic, sulfur-containing compounds which accumulate mainly in roots and hypocotyls. They are characterized by a 5-membered ring which contains an S atom. A sulfhydryl group, which originates from the amino acid cysteine, participates in the formation of the ring. Important representatives of the thiophenes which are also formed in Tagetes are, for example, BBT (5-(but-3-en-1-ynyl)-2,2′-bi-thienyl), BBTOAc (5-(4-acetoxy-1-butynyl)-2,2′-bithienyl), BBTOH (5-(4-hydroxy-1-butynyl)-2,2′-bithienyl) and α-T (2,2′:5′,2″-terthienyl).

The petals of Tagetes contain 9-22% of flavonoids and approx. 27% of carotenoids, in which β-carotene accounts for 0.4%, cryptoxanthin esters for 1.5% and xanthophyll esters for 86.1% (Benk et al., Riechst, Aromen und Koerpen, 26 (1976), 216-221).

According to an estimate from 1975, over 2000 different flavonoids exist. Some important representatives are, for example, the anthocyanins with 250 known structures, the chalcones with 60, the aurones with 20 and the flavones with 350 known structures, all of which have coloring properties. Flavonols with 350 and the isoflavonoids with 15 representatives impart to the plants properties such as phagoprotection or have a toxic effect on fungi.

Carotenoids belong to the large group of the terpenoids. Most of them are tetraterpenes. Carotenoid-hydrocarbons are also termed carotenes, and their oxidized derivatives are the xanthophylls. Carotenoids are essential components in photosynthetically active membranes of all plants, algae and cyanobacteria. They are found in the pigment systems (light traps) of the chloroplasts and participate in the process of the primary light absorption and photon channeling of photosynthesis. Moreover, they also act as light receptors in a series of other light-induced processes in the plant. The yellow color of many flowers is due to carotenoid-containing, usually chlorophyll-free, chromoplasts. Plant carotenoids also act as precursors for the biosynthesis of the plant growth regulator abscisic acid and of vitamin A, which is important for human and animal nutrition. It is increasingly of interest as a potential anticancer agent and is employed as colorant in the cosmetics and food industries. Carotenoids from dried and pulverized Tagetes petals are already being used as poultry feed additive for intensifying the color of chicken egg yolk.

There is currently a great economic interest in increasing, or improving, the carotenoid content and the carotenoid composition in plants.

The limited genetic variability in known Tagetes varieties greatly limits the possibilities of improving these varieties with the aid of traditional biolological plant breeding methods.

Genetic engineering methods allow the directed transfer of foreign genes into the plant genome. This process is termed transformation, and the resulting plants are termed transgenic. The basic prerequisite for the generation of transgenic plants is the availability of a suitable transformation system, the presence of a selectable marker, the identification of successfully transformed plant cells, and the regeneration of the transformed cells into intact fertile plants.

A variety of processes is currently available for the transformation. The most frequently employed method of transforming dicotyledonous plants is the Agrobacterium-tumefaciens-mediated gene transfer. This method exploits the natural ability of the soil bacterium to integrate genetic material into the plant genome. Other suitable methods are, for example, protoplast transformation by polyethylene-glycol-induced DNA uptake, electroporation, sonication or microinjection, and the transformation of intact cells or tissue by micro- or macroinjection into tissue or embryos, tissue electroporation, incubation of dry embryos in DNA-containing solution, vacuum infiltration of seeds, and the biolistic gene transfer.

The use of Agrobactgerium tumefaciens for the transformation of plants using tissue culture explants has been described by Horsch et al. (Science 228(1985), 1229-1231), Fraley et al. (Proc. Natl. Acad. Sci. USA 80(1983), 4803-4807) and Bevans et al. (Nature 304(1983), 184-187). Many strains of Agrobactgerium tumefaciens are capable of transferring genetic material, including into Tagetes species, such as, for example, the strains EHA101[pEHA101], EHA105[pEHA105], LBA4404[pAL4404], C58C1[pMP90] and C58C1[pGV2260]. Strain EHA101[pEHA101] has been described by Hood et al. (J. Bacteriol. 168(1986), 1291-1301), strain EHA105[pEHA105] by Hood et al. (Transgenic Research 2 (1993), 208-218), strain LBA4404[pAL4404] by Hoekema et al. (Nature 303(1983), 179-181), strain C58C1[pMP90] by Koncz and Schell (Mol. Gen. Genet. 204(1986), 383-396) and strain C58C1[pGV2260] by Deblaere et al. (Nucl. Acids Res. 13(1985), 4777-4788).

The agrobacterial strain employed for the transformation contains in addition to its disarmed Ti-plasmid a binary plasmid with the T-DNA to be transferred which, as a rule, contains a gene for selecting the transformed cells and the gene to be transferred. Both genes must be equipped with transcriptional and translational initiation and termination signals. The binary plasmid can be transferred into the agrobacterial strain, for example, by electroporation or other transformation methods (Mozo & Hooykaas, Plant Mol. Biol. 16(1991), 917-918). As a rule, coculture of the plant explants with the agrobacterial strain takes two to three days.

Foreign genes can be expressed in a constitutive, inducible (by biotic and abiotic factors), tissue-specific or development-specific fashion. A relatively constitutive expression is achieved, for example, in plants by the cauliflower mosaic virus 35S promoter, and this expression has been described by Shewmaker et al. (Virology 140(1985), 281-288) and Gardner et al. (Plant Mol. Biol. 6(1986), 221-228).

Selectable marker genes which can be used are, for example, the bialophos resistence gene (bar), the kanamycin or G418 resistence gene (NPTII) and the DOG^{R}1 gene. The bialophos resistence gene was originally isolated from Streptomyces hygroscopicus. It encodes a phosphinothricin acetyltransferase (PAT), which acetylates the free amino group of phosphinothricin (PPT), thus detoxifying PPT (de Block et al., EMBO J. 6(1987), 2513-2518). The NPTII gene encodes a neomycin phosphotransferase, which reduces the inhibitory action of kanamycin, neomycin, G418 and paromomycin by a phosphorylation reaction. The gene DOG^{R}1 has been isolated from the yeast Saccharomyces cerevisiae (Sonnewald and Ebneth, EP 0 807 836). It encodes a 2-deoxyglucose-6-phosphate phosphatase, which imparts resistence to 2-DOG (Randez-Gil et al., Yeast 11(1995), 1233-1240).

Despite its interesting biotechnological properties, the genus Tagetes has previously infrequently been the subject of recombinant modifications. The prerequisite for a recombinant improvement of Tagetes is a good regeneration system. Regeneration by organogenesis using leaves as starting explant was developed by Ketel (Physiol. Plant. 93 (1986), 298-304), Khotari and Chandra (Hortscience 19(1984a), 703-705) and [lacuna] (J. Plant Physiol. 122(1986), 235-241). Khotari and Chandra also described the regeneration of plants from immature, unfertilized flower disks (J. Plant Physiol. 117(1984b), 105-108). Hypocotyls can also be used for regeneration (Belarmino et al., Jpn J. Breed 42(1992), 835-841).

The transformation and establishment of Tagetes laxa root cultures has been described by Talou et al. (Planta Médica 60 (1994), 260-262), but transformation methods with a subsequent regeneration into transgenic fertile plants have not been described as yet for the genus Tagetes.

Other known genera of the family Asteraceae are, for example, Taraxacum with the known species Taraxacum officinale (dandelion), Dahlia, Helianthus with the known species Helianthus annuus (sunflower), Aster, Calendula (calendula), Carduus (simple pappus), Cichorium and the like. With the exception of sunflower, no, or only insufficient, in-vitro techniques or other techniques which would be suitable for the transformation of this species exist for all of the genera of this family. Even though the individual representatives of the Asteraceae family are morphologically relatively uniform, in-vitro regeneration and transformation techniques cannot be generalized, so that the use of transformation protocols which have been developed for, for example, sunflower, is not possible for other genera such as Tagetes.

It is an object of the present invention to provide a method for the generation of transgenic fertile plants of the genus Tagetes.

The method according to the invention allows for the first time the possibility of transformed plant cells of the genus Tagetes to be regenerated into fertile transgenic plants.

The method for the generation of stably transformed Tagetes plants is composed of the following steps: (a) growing the starting plant to be transformed, and obtaining the suitable explant, (b) transferring DNA sequences into plant cells, (c) selecting transformed plant cells, and (d) regenerating these transformed plant cells into fertile transgenic plants.

Both the direct and the indirect gene transfer are suitable transformation methods. The agrobacterium-mediated transformation using a wide range of starting explants, such as, for example, cotyledons, leaves, hypocotyls, shoots, roots, callus, mature and immature seeds or floral tissue can successfully be employed. The gene transfer can be effected both by simple cocultivation/incubation or wetting with the agrobacterial strain and by a supporting vacuum infiltration of the explants with the bacterial culture in question. The use of feeder cultures as aids may be advantageous in the process. Each agrobacterial strain which contains a Ti- or Ri-plasmid with the genetic informations required for the transfer is suitable as vector for the transformation. Suitable agrobacterial strains are EHA101[pEHA101], EHA105[pEHA105], LBA4404[pAL4404], C58C1[pMP90] and C58C1[pGV2260]. Viral vectors also seem to be suitable for the transformation of Tagetes. Other methods for transferring genetic material into Tagetes are, for example, the polyethylene glycol (PEG)-mediated protoplast transformation, the electroporation, the sonication or microinjection, and the transformation of intact cells or tissue by micro- or macroinjection into tissue or embryos, tissue electroporation, incubation of dry embryos in DNA-containing solution, the vacuum infiltration of seeds, see also Eds. Galun, E. and Breiman, A.

In: Transgenic Plants, Imperial College Press, 1997. The use of the particle gun for bombarding a wide range of explants such as, for example, leaves and callus, is also possible.

All gene transfer vectors which contain the border sequences necessary for transferring the T-DNA (left and right border, LB and RB respectively), carry one or more selectable marker or reporter genes under the control of suitable promoters and terminators and/or contain further useful genes or target genes, also under the control of suitable transcriptional and translational regulatory units, can be employed for the agrobacteria-mediated transformation. Feasible selectable marker or reporter genes are not only the genes bar/PAT, NPTII and DOG^{R}1, but also, for example, the following: the chloramphenicol acetyltransferase (CAT) gene from the transposon Tn9, the octopine synthase and nopalin synthase genes, both from the T region of the plasmid pTi, the E. coli hygromycin phosphotransferase gene, the Salmonella typhimurium aroA gene, which encodes EPSP synthase and thus mediates resistence to glyphosate, the E. coli glucuronidase gene, the luciferase gene and the GFP gene in its presently available variants, which encodes an autofluorescent—originally exclusively green fluorescent—protein. Moreover, all other genes which encode resistance to antimetabolites, antibiotics or herbicides can be used as selectable marker genes, see also Wilmink, A. and Dons, J. J. M. (Plant Mol. Biol. 11(1993), 165-185). Positive selection systems can also be used, for example the acquisition of the capability of utilizing sugar, as described in WO 94/20627.

The transfer of DNA sequences into the Tagetes plant to be transformed or explants thereof is preferably effected by Agrobacterium-tumefaciens-mediated gene transfer.

The regeneration medium according to the invention has a sucrose concentration of 0.5-5% sucrose, preferably 1-3% sucrose. If appropriate, sucrose may also be replaced by other sugars—glucose or fructose, inter alia.

The regeneration medium according to the invention furthermore comprises growth regulators such as auxins and/or auxin conjugates, and cytokinins and/or cytokinin conjugates. Suitable auxins are both natural and synthetic auxins. The natural auxin is indoleacetic acid (IAA), examples of synthetic auxins are 3-indolebutyric acid (IBA), 1-naphthylacetic acid (NAA) and the herbicide 2,4-dichlorophenoxyacetic acid (2,4-D). Other herbicides which act as auxins are also feasible as growth regulators. Auxin conjugates are compounds of auxin (IAA) with aspartic acid, glucose, myoinositol and others. In the case of the cytokinins, again, natural cytokinins such as, for example, zeatin and synthetic components, such as 6-benzylaminopurine (BAP) and 6-furfurylaminopurine (kinetin) may also be employed. Zeatin riboside is frequently employed as cytokinin conjugate. The auxins and the cytokinins can be employed in each case as individual components, but also as auxin or cytokinin mixtures. The concentration of the individual growth regulators is 0.05-10 mg/l, preferably 0.1-5 mg/l.

In a particular embodiment, the culture medium is first supplemented with the growth regulators benzylaminopurine, and indoleacetic acid for culturing the explants during and after cocultivation with the bacteria (phase 1). After cocultivation, the explants are transferred to the same basal medium which additionally contains an antibiotic, such as, for example, carbenicillin, timentin, ticarcillin, cefotaxime or β-bactyl for suppressing bacterial growth, and the actual selective agent for concentrating the transgenic cell material. Transfer to fresh medium of the same composition takes place in each case after 14 days, until shoot buds and small shoots have developed (duration: approx. 4-6 weeks). Subsequently, a medium which contains the growth regulators 3-indolebutyric acid and the gibberellic acid GA₃ instead of benzylaminopurine and indoleacetic acid is used for the further shoot elongation and root development (phase 2).

The regeneration method according to the invention for the generation of transgenic fertile plants of the genus Tagetes can be applied, inter alia, to the species Tagetes (T.) patula, T. erecta, T. laxa, T. minuta, T. lucida, T. argentina cabrera, T. tenuifolia, T. lemmonii or T. bipinata.

### EXAMPLE 1

### Construction of the Binary Plasmid pPTGI

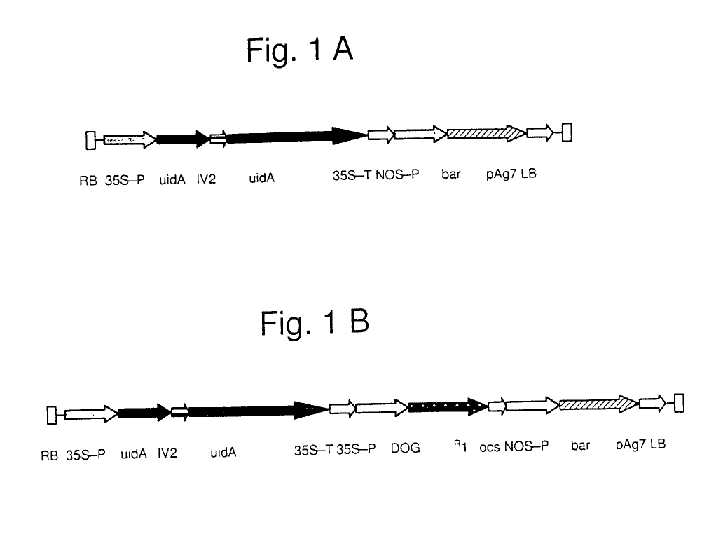

To construct the binary plasmid pPTGI (FIG. 1A), the plasmid pGPTV-Bar (Becker, D. et al., Plant Mol. Biol. 20(1992), 1195-1197) was used. The promoter-less GUS gene of this plasmid was removed by cleaving the plasmid with the restriction enzymes EcoRI and SmaI. The overhangs have subsequently been filled up using Klenow polymerase and the fragment ligated with a PstI fragment which contains the glucuronidase gene with an intron (GUS-IV2) (Vancanneyt, Mol. Gen. Genet. 220 (1990), 245-250) and which was treated in the same manner.

To construct the binary plasmid pPTGIDOG, the DOG^{R}1 gene with its adjacent 35S promoter region and ocs terminator region was amplified by means of PCR. The pBinAR-DOG^{R}1 construct, which has been described by Sonnewald and Ebneth, EP0807836, was used as template sequence. The two primers 35SXbaI and OcsXbaI have been employed for the amplification. 35SXbaI is homologous to nucleotides 1 to 24 of the 35S promoter of plasmid pBinAR (Höfgen and Willmitzer (Plant Science) 66 (1990), 221-230) and additionally comprises an XbaI recognition region at the 5′ end (5′-ATTCTAGACATGGAGTCAAAGATTCAAATAGA-3′). OcsXbaI is homologous to the last 24 nucleotides of the ocs terminator region of plasmid pBinAR and comprises an additional XbaI restriction site (5′-ATTCTAGAGGACAATCAGTAAATTGAACGGAG-3′). The amplified fragment was freed from its overhanging simplex ends using Klenow polymerase, ligated with HindIII linkers and cloned into plasmid pBS+ (Stratagene, Calif., USA). Following sequence analysis, the complete fragment, which contained the 35S promoter, the DOG^{R}1 gene and the ocs terminator region, has been cloned as HindIII fragment into the HindIII cut vector PPTGI. FIG. 1 shows the gene maps of the T-DNAs of the two binary plasmids pPTGI (5007 bp) (FIG. 1A) and pPTGI-DOG^{R}1 (6209 bp) (FIG. 1B), both of which were used for the transformation of Tagetes. The abbreviations have the following meanings:

| | |
|---|---|
| 35S-P: | CaMV 35S promoter |
| 35S-T: | CaMV 35S terminator |
| NOS-P: | nopaline synthase promoter |
| uidA: | β-glucuronidase gene |
| bar: | phosphinothricin acetyltransferase gene |
| LB: | left border |
| RB: | right border |
| IV2: | 2^{nd} intron of gene ST-LS1 |
| pAg7: | Gene 7 poly(A) signal |
| DOG^{R}1: | 2-DOG-phosphate phosphatase gene of Saccharomyces |
| | cerevisiae S288c |
| ocs: | octopine synthase terminator |

### EXAMPLE 2

### Determination of Phosphinothricin (PPT) Toxicity

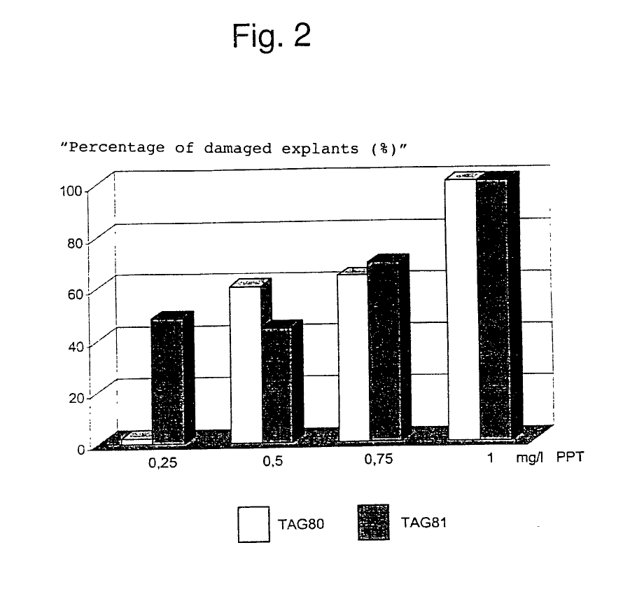

Leaves of Tagetes patula plants of genotypes TAG 80 and TAG 81 (Genbank, IPK Gatersleben) which were established in vitro were cut transversely to the central vein into disks 10-60 mm² in size and placed on MS medium supplemented with 2% sucrose, 3 mg/l benzylaminopurine (BAP), 1 mg/l indoleacetic acid (IAA) and different PPT concentrations. Incubation was for 2 weeks in a 16/8 hour light/dark photoperiod at approx. 50 μE/m²s and at from 21 to 24° C. Then, the number of damaged explants was recorded with reference to the total number of tested explants (FIG. 2). In both Tagetes patula genotypes tested, concentrations starting at 1 mg/l PPT led to the complete destruction of all explants, which is why this concentration was used for the selection after cocultivation.

### EXAMPLE 3

### Tagetes erecta Transformation

In vitro seedling explants of Tagetes erecta TAG 76 (Genbank, IPK, Gatersleben) were used as primary target material. For surface sterilization, the seed was incubated for 5 minutes in 70% ethanol and subsequently washed thoroughly with sterile distilled water. Thereafter, the seeds were dried on filter paper, placed on solid MS medium (Murashige and Skoog (1962) Physiol. Plant. 15, 473-497) and incubated for 3 to 12 weeks in a 16/8 hour light/dark photoperiod at approx. 50 μE/m²s and at from 21 to 24° C. All leaves (with the exception of cotyledons) formed during this time were harvested and cut transversely to the central vein. The leaf explants thus obtained, which had a size of from 10 to 60 mm^{2,} were kept for not longer than 2 hours during the preparation in liquid MS medium at room temperature.

The Agrobactgerium tumefaciens culture EHA105[pPTGI] was grown overnight for 16 to 20 hours at 28° C. in YEB (0.1% yeast extract, 0.5% beef extract, 0.5% peptone, 0.5% sucrose, 0.5% magnesium sulfate×7 H₂O) with 25 mg/l kanamycin by inoculation with a single colony. The bacterial suspension was subsequently harvested by centrifugation for 10 minutes at 6000 g and resuspended in liquid MS medium in such a manner that an OD of approx. 0.3 to 0.8 resulted.

Immediately prior to cocultivation, the MS medium in which the leaves have been stored has been replaced by the bacterial suspension. Incubation of the leaflets in the agrobacterial suspension was done for 30 minutes at room temperature with gentle shaking. The infected explants were subsequently placed on MS medium supplemented with 2% sucrose and 3 mg/l benzylaminopurine (BAP) and 1 mg/l indoleacetic acid (IAA) and solidified with 0.8% Plant Agar (Duchefa, N L). The application of the growth regulators corresponds to a combination developed by Kothari and Chandra (Hort-Science 19 (1984), 703-705). The orientation of the leaves on the medium is of no importance. Cultivation of the explants took place for 6 days under the same conditions as described for the germination of the Tagetes seeds. The cocultured explants were subsequently transferred to fresh MS medium with identical growth regulators, this second medium additionally containing 250 mg/l β-bactyl and 1 mg/l PPT.

At intervals of 14 days, the explants were transferred to fresh medium, until shoot buds and small shoots developed, and these were subsequently transferred to the same basal medium inclusive of β-bactyl and PPT, but with an altered combination of growth regulators, viz 0.5 mg/l IBA and 0.5 mg/l GA₃, in order to root. Rooted shoots were transferred into the greenhouse.

### EXAMPLE 4

### Tagetes patula Transformation

In-vitro-established Tagetes patula TAG80 plants (Genbank, IPK, Gatersleben) served as target material for the transformation. They were obtained or propagated routinely by shoot tip propagation using solid MS medium supplemented with the growth regulators IBA (0.5 mg/l) and GA₃ (0.5 mg/l). Leaves of these plants were cocultured for 4, 6 and 8 days with the Agrobacterium tumefaciens strain EHA105[pPTGI-DOG^{R}1]. The experimental set-up and the regeneration conditions used were analogous to those described in Example 3. Candidate transgenic shoot regenerates were rooted in the presence of 1 mg/l PPT.

### EXAMPLE 5

### Test for Transgenicity

A. Detection of Glucuronidase

Leaves and root segments of the regenerated in vitro plants were subjected to a qualitative glucuronidase (GUS) enzyme detection by infiltrating them for 1 minute in vacuo with the GUS substrate 5-bromo-4-chloro-3-indolyl-β-D-glucuronic acid (X-GlcA) in a 100 mM sodium phosphate buffer, pH 7.0, which contained 10 mM EDTA, 0.1% Triton X100 and 10 mM DTT, and subsequently incubating them for approximately 15 hours at 37° C. The explants were then decolored with 70% ethanol at room temperature. Leaves and roots showed an intense blue coloration, which proves that the reporter gene is expressed in Tagetes.

B. Genomic PCR Analysis

Genomic DNA was isolated from the candidate transgenic plants to be tested and from 3 untransformed wild-type plants using the following method: approx. 100 mg of leaf material were frozen in liquid nitrogen in a reaction vessel and subsequently disrupted with a homogenizer. The material was extracted in 900 μl of extraction buffer (100 mM Tris-HCl, pH 8.0, 500 mM NaCl, 50 mM Na₂EDTA, 10 mM mercaptoethanol). Following addition of 100 μl of 20% SDS solution, the material was mixed and incubated for 10 minutes at 65° C. Addition of 200 μl of 5 M potassium acetate solution was followed by incubation for 20 minutes at 4° C. in an ice bath. The precipitate formed during this time was removed by centrifugation at 12,000 g for 15 minutes, and the DNA in the supernatant was precipitated in the course of 30 minutes by addition of 800 μl of ice-cold isopropanol at −20° C. The samples were spun to collect the DNA, which was subsequently washed with 70% ice-cold ethanol, dried in the air and resuspended in 25 μl of 10 mM Tris-HCl, pH 8.0, 1 mM EDTA (TE) with 0.2 mg/ml RNase A. 3 μl of the DNA isolated thus were employed in the PCR reaction.

The DOG^{R}1 gene comprises 741 bp and was amplified by using the two primers DOG^{R}1-1 and DOG^{R}1-2. DOG^{R}1-1 is homologous to nucleotides 1 to 26 of the DOG^{R}1 gene and contains 10 additional nucleotides, which encompass the restriction recognition sites BamHI and NcoI (5′-ATGGATCCCCATGGCAGAATTTTCAGCTGATCTATG-3′). DOG^{R}1-2 is homologous to nucleotides 720 to 741 of the DOG^{R}1 gene and contains 11 additional nucleotides, which comprise the restriction recognition site SalI (5′-ATGTCGACTACTCAGGCCCTTGTCAAAGGGTTG-3′). The PCR reaction was carried out as specified by the manufacturer Takara (Japan). The following incubation steps were performed:

1. 94° C.-4 min

2. 94° C.-15 sec

3. 58° C.-30 sec

4. 72° C.-1 min

repeat steps 2-4 4 times

5. 94° C.-15 sec

6. 56° C.-30 sec

7. 72° C.-1 min

repeat steps 5-7 4 times

8. 94° C.-15 sec

9. 52° C.-30 sec

10. 72° C.-1 min

repeat steps 8-10 24 times

11. 72° C.-10 min

12. 4° C.-end of the reaction

Following the PCR reaction, 5 μl of the individual samples have been treated with 5 μl of stop solution (10 mM Tris HCl, pH 8.0, 50% glycerol, 0.05% SDS, 0.2% xylene cyanole) and separated in a 1% agarose gel. After the electrophoretic separation at 100 V had been performed for 30 minutes, the gel was evaluated under UV light. Table 1 contains the results of the analyses regarding the trangenicity of candidate Tagetes patula transformants. All these plants were regenerated in the presence of 1 mg/l PPT. Only those plants which were capable of repeatedly forming roots in the presence of the herbicide were used in the PCR analysis. The DOG^{R}1 gene was detected in all these plants. Surprisingly, GUS activity in the leaves was only detectable in few plants.

TABLE 1
Analysis of candidate transgenic Tagetes patula
plants transformed with the binary plasmid
pPTGI-DOG^{R}1 (see Example 4).

| | Coculture | | | Genomic PCR |
|---|---|---|---|---|
| Plant | time in | Resistance/ | GUS | (DOG^{R}1 |
| No. | days | re-rooting^{a)} | activity^{b)} | primer)^{c)} |
| 1 | 6 | − | − | n.d. |
| 2 | 6 | − | − | n.d. |
| 3 | 6 | − | − | n.d. |
| 4 | 6 | + | − | + |
| 5 | 6 | (+) | − | + |
| 6 | 6 | (+) | − | + |
| 7 | 6 | (+) | − | + |
| 8 | 6 | − | − | n.d. |
| 9 | 6 | ++ | − | + |
| 10 | 6 | ++ | − | + |
| 11 | 6 | ++ | − | + |
| 12 | 6 | − | − | n.d. |
| 13 | 6 | − | − | n.d. |
| 14 | 6 | − | − | n.d. |
| 15 | 6 | + | − | + |
| 16 | 6 | + | − | + |
| 17 | 6 | ++ | (+) | + |
| 18 | 6 | − | − | n.d. |
| 19 | 6 | (+) | − | − |
| 20 | 4 | − | − | n.d. |
| 21 | 4 | − | − | n.d. |
| 22 | 4 | ++ | (+) | + |
| 23 | 4 | + | − | + |
| 24 | 8 | − | − | n.d. |
| 25 | 8 | − | − | n.d. |
| 26 | 8 | (+) | − | + |
| 27 | 8 | ++ | (+) | + |
| 28 | 8 | − | − | n.d. |
| 29 | 8 | (+) | (+) | + |
| 30 | 8 | ++ | (+) | + |

Key to the symbols:
^{a)}− In the presence of 1 mg/l PPT, the shoot of this plant is incapable of forming roots, only shows weak growth and dies.
(+) The shoot only shows poor root formation ability. Plant growth is poor.
+ The shoot shows pronounced root formation. Plant growth is normal.
++ The shoot forms a vigorous root system within a short period. Plant grows without adverse effects in the presence of 1 mg/l PPT.
^{b)}− No GUS activity detectable.
(+) Weak GUS activity can be observed in the form of small, blue dots on the leaf surface.
^{c)}n.d. not determined
+ Following PCR reaction and subsequent separation by gel electrophoresis, a pronounced band of approx. 750 bp can be detected. This band was not detectable in control reaction in which the genomic DNA of Tagetes wild-type plants has been employed.

### EXAMPLE 6

### Progeny Analysis

To carry out the progeny analysis, the transgenic Tagetes erecta TAG 76 plant No. 2 was employed. The generation of this plant was described in Example 3.

Seed of this plant and of an untreated wild-type plant was sown into plant dishes (approx. 30×60 cm) and cultured at approx. 25° C. under greenhouse conditions. After 12 days, the seedlings had formed approx. 3 to 4 leaves and obtained a height of approx. 5 to 10 cm. They were then sprayed three times at one-day intervals with a solution of the herbicide Basta, diluted 1:1000. Four days after the last spray application of the herbicide, the experiment was evaluated. All 83 wild-type seedlings which had been sprayed showed massive damage or had already died. Among the seedling progeny of transgenic plant No. 2, 134 of the 190 plants which had been sprayed showed no damage whatsoever, and 56 were damaged or died. This segregation ratio of approx. 2.5 resistant plants: 1 sensitive plant suggests Mendelian inheritance, which should ideally be 3:1.

We claim:

1. A method for the generation of stably transformed fertile plants of the genus Tagetes, which comprises the following steps:
(a) growing the starting plant to be transformed and obtaining the suitable explant,
(b) transferring DNA sequences into plant cells,
(c) selecting transformed plant cells,
(d) regenerating fertile transgenic plants.

2. A method as claimed in claim 1, wherein DNA sequences are transferred into the plant to be transformed or explants thereof by means of agrobacteria.

3. A method as claimed in claim 2, wherein leaves are used as explants.

4. A method as claimed in either of claims 2 and 3, wherein cocultivation with agrobacteria takes place for 2-8 days.

5. A method as claimed in any of claims 2 to 4, wherein the Agrobacterium species used is Agrobactgerium tumefaciens.

6. A method as claimed in any of claims 2 to 5, wherein a medium is used during the cocultivation which contains growth regulators.

7. A method as claimed in claim 1 or 2, wherein herbicide, antibiotic or antimetabolite resistances are employed for selecting the transformed cells or positive selection methods are used.

8. A method as claimed in claim 7, wherein phosphinothricin (PPT) is employed for the selection.

9. A method as claimed in any of claims 1 to 8, wherein two different combinations of growth regulators are used for the regeneration into intact transgenic plants of the genus Tagetes, benzylaminopurine and indoleacetic acid being employed in phase 1 and indolebutyric acid and gibberellic acid GA₃ in phase 2.

10. A method as claimed in any of claims 1 to 9, wherein the species Tagetes erecta or Tagetes patula is transformed.

11. A transformed Tagetes plant generated by a method as claimed in any of claims 1 to 10.

12. A transformed seed of a Tagetes plant obtained from a plant as claimed in claim 11.


SEQUENCE LISTING (4 sequences)

SEQ ID NO: 1 - DNA, 32 bp, Artificial Sequence
  Description of Artificial Sequence primer with XbaI recognition region
attctagacatggagtcaaagattcaaataga

SEQ ID NO: 2 - DNA, 32 bp, Artificial Sequence
  Description of Artificial Sequence primer with XbaI recognition site
attctagaggacaatcagtaaattgaacggag

SEQ ID NO: 3 - DNA, 36 bp, Artificial Sequence
  Description of Artificial Sequence primer with BamHI and NcoI recognition sites
atggatccccatggcagaattttcagctgatctatg

SEQ ID NO: 4 - DNA, 33 bp, Artificial Sequence
  Description of Artificial Sequence primer with SalI site
atgtcgactactcaggcccttgtcaaagggttg